(19) United States
(12) Patent Application Publication
(10) Pub. No.: US 2021/0030849 A1
(43) Pub. Date: Feb. 4, 2021

(54) LIQUID COMPOSITION FOR ORAL USE CONTAINING COLLAGEN PEPTIDE, AND METHOD FOR IMPROVING FLAVOR OF LIQUID COMPOSITION FOR ORAL USE INCLUDING COLLAGEN PEPTIDE

(71) Applicant: SUNTORY HOLDINGS LIMITED, Osaka-shi, Osaka (JP)

(72) Inventors: Megumi Okada, Soraku-gun (JP); Takako Imao, Soraku-gun (JP)

(73) Assignee: SUNTORY HOLDINGS LIMITED, Osaka-shi, Osaka (JP)

(21) Appl. No.: 16/967,838

(22) PCT Filed: Jan. 30, 2019
(86) PCT No.: PCT/JP2019/003161

§ 371 Date: Aug. 6, 2020

(30) Foreign Application Priority Data
Feb. 9, 2018 (JP) 2018-022437

(51) Int. Cl. A61K 38/39; A61K 47/36; A61K 9/00

(52) U.S. Cl. CPC A61K 38/39; A61K 9/0095; A61K 47/36

(57) ABSTRACT

The present invention provides a technique to reduce the unpleasant odor, particularly medium-like odor, and bitterness of a liquid oral composition containing low molecular weight collagen peptides. The present invention relates to a liquid oral composition containing collagen peptides having an average molecular weight of 300 to 2000 and welan gum.

### TECHNICAL FIELD

The present invention relates to a liquid oral composition containing collagen peptides. The present invention also relates to a method of improving the flavor of a liquid oral composition containing collagen peptides.

### BACKGROUND ART

Collagen peptides have been revealed to have various functions such as making the skin beautiful (e.g., improving moisture-retaining properties and elasticity of the skin) and improving blood fluidity, and are nowadays contained in many beverages, foods, cosmetics, and the like.

Benefiting from collagen peptides requires continuous intake of the collagen peptides. However, many collagen peptides which are degraded proteins have a distinct odor such as a fishy odor. Such an odor is particularly strong when collagen peptides are added to beverages. As a method of suppressing the odor of a beverage containing collagen peptides, Patent Literature 1 discloses use of a specific component such as maltol and a specific flavonoid such as catechin in a liquid oral composition containing proteolytic peptides such as collagen peptides.

### CITATION LIST

### Patent Literature

Patent Literature 1: JP 2010-222287 A

### SUMMARY OF INVENTION

### Technical Problem

Low molecular weight collagen peptides are known to have good absorbability in the body. The present inventors designed a liquid oral composition containing low molecular weight collagen peptides having an average molecular weight of 300 to 2000, and they found that a liquid oral composition containing such low molecular weight collagen peptides has a strong distinct unpleasant odor, particularly medium-like odor (peptone-like odor), and a strong bitterness which are attributable to the collagen peptides. Yet, no studies have been made on techniques to reduce the medium-like odor and bitterness attributable to collagen peptides in liquid oral compositions such as beverages. In order to enable a continuous intake of low molecular weight collagen peptides, it is desirable to develop a novel technique capable of reducing the medium-like odor and bitterness attributable to collagen peptides.

The present invention aims to provide a technique to reduce the unpleasant odor, particularly medium-like odor, and bitterness of a liquid oral composition containing low molecular weight collagen peptides.

### Solution to Problem

As a result of extensive studies to solve the above problem, the present inventors found that adding welan gum to a liquid oral composition containing collagen peptides having an average molecular weight of 300 to 2000 can reduce (suppress) the unpleasant odor, particularly medium-like odor, and bitterness attributable to the collagen peptides in the liquid oral composition.

Specifically, the present invention relates to the following liquid oral composition and the like.

(1) A liquid oral composition containing: a collagen peptide having an average molecular weight of 300 to 2000; and welan gum.

(2) The liquid oral composition according to (1) above, wherein the amount of the collagen peptide is 600 to 20000 mg/100 mL.

(3) The liquid oral composition according to (1) or (2) above, wherein the weight ratio of the collagen peptide to the welan gum (collagen peptide/welan gum) is 3 to 70.

(4) The liquid oral composition according to any one of (1) to (3) above, wherein the amount of the welan gum is 50 to 400 mg/100 mL.

(5) The liquid oral composition according to any one of (1) to (4) above, wherein the viscosity is 30 to 500 mPa·s.

(6) The liquid oral composition according to any one of (1) to (5) above, further containing a proteoglycan and/or elastin peptide.

(7) The liquid oral composition according to any one of (1) to (6) above, which is a beverage.

(8) A method of improving the flavor of a liquid oral composition, the method including: adding welan gum to a liquid oral composition containing a collagen peptide having an average molecular weight of 300 to 2000.

### Advantageous Effects of Invention

The present invention can reduce the unpleasant odor, particularly medium-like odor, and bitterness attributable to low molecular weight collagen peptides in a liquid oral composition containing such collagen peptides.

### DESCRIPTION OF EMBODIMENTS

### <Liquid Oral Composition>

The liquid oral composition of the present invention contains collagen peptides having an average molecular weight of 300 to 2000 and welan gum.

The collagen peptides having an average molecular weight of 300 to 2000 used in the present invention are hereinafter also simply referred to as “collagen peptides”.

### <Collagen Peptides>

The collagen peptides used in the present invention has an average molecular weight of 300 to 2000.

The present invention can reduce the unpleasant odor, particularly medium-like odor (peptone-like odor), and bitterness attributable to the low molecular weight collagen peptides in the liquid oral composition containing such collagen peptides. Thus, the present invention can reduce the medium-like odor and bitterness in the liquid oral composition containing such collagen peptides and improve the flavor of the composition for easier intake.

Collagen peptides having a smaller average molecular weight tend to have a stronger medium-like odor. In addition, collagen peptides having a smaller average molecular weight tend to have a stronger bitterness due to a higher degree of degradation to amino acids. The average molecular weight of the collagen peptides is preferably 1800 or less, more preferably 1500 or less, for allowing the effect of reducing the medium-like odor and bitterness to be more sufficiently exerted. In one embodiment, the average molecular weight of the collagen peptides may be 1000 or less, or 800 or less. In order to more sufficiently reduce the bitterness, the average molecular weight of the collagen peptides is preferably 350 or more, more preferably 400 or more. Herein, the range may be any combination of any upper limit and any lower limit. In one embodiment, the average molecular weight of the collagen peptides is preferably 350 to 1800, more preferably 400 to 1500, still more preferably 400 to 1000, particularly preferably 400 to 800.

Herein, the average molecular weight of the collagen peptides is the weight average molecular weight. Herein, the average molecular weight of the collagen peptides means the value measured by a relative molecular mass measurement method in Chinese National Standards (GB standards) GB/T 22729-2008: oligopeptides powder of marine fish. Yet, substitution products are used as reagents for M, 451 and M, 189.

In this method, substances whose molecular weights are known such as cellular pigment C (cytochrome, M, 6500), Trasylol (aprotinin, M, 12500), Bacillus (bacitracin, M, 1450), glycine-glycine-tyrosine-arginine (M, 451), and glycine-glycine-glycine (M, 189) are measured in advance under the same conditions to obtain a relative molecular mass calibration curve showing a relationship between retention time and logarithm of relative molecular weight. The average molecular weight of the collagen peptides is calculated based on the calibration curve. The average molecular weight herein means the weight average molecular weight calculated in terms of each standard substance according to this method.

The collagen peptides used in the present invention can be obtained by hydrolysis of collagen or modified collagen such as gelatin with an enzyme, acid, alkali, or the like. The source and production method of the collagen peptides are not limited. Artificially synthesized collagen peptides can also be used. One type of collagen peptides may be used alone or two or more types of collagen peptides may be used in combination.

Collagen or gelatin as a raw material of the collagen peptides may be one from bovine, swine, chicken, fish, or the like. One or more of these can be used as raw materials. In one embodiment, collagen from fish is preferred. Fish may be saltwater fish or freshwater fish. Examples include tuna (yellowfin), shark, cod, olive flounder, righteye flounder, sea bream, tilapia, salmon, and catfish.

Any enzyme may be used to prepare the collagen peptides as long as the enzyme can cleave peptide bonds of collagen or gelatin. Examples include collagenase, papain, bromelain, actinidine, ficin, cathepsin, pepsin, chymosin, trypsin, and enzymatic preparations in which these enzymes are mixed. The acid may be, for example, hydrochloric acid, sulfuric acid, nitric acid, or the like. The alkali may be, for example, sodium hydroxide, calcium hydroxide, or the like.

In the present invention, an aqueous solution of hydrolyzed collagen peptides may be used as is or a hydrolyzed collagen peptide powder obtained by drying or the like may be used. Alternatively, the aqueous solution subjected to a usual purification treatment may be used in the form of an aqueous solution, a powder, or the like.

Commercially available collagen peptides may be used. Collagen peptides having the average molecular weight described above can be used. Examples of such commercial products include “Collapep PU” (Nitta Gelatin Inc.), “TYPE-S” (Nitta Gelatin Inc.), and “HACP” (Jellice Co., Ltd.).

In one embodiment, it is preferred that the collagen peptides in the present invention contain a large amount of Pro-Hyp (prolyl-hydroxyproline (hereinafter “PO”)) and/or Hyp-Gly (hydroxyprolyl-glycine (hereinafter “OG”)) which are dipeptides. It is more preferred that the collagen peptides contain a large amount of PO and OG. Collagen peptides containing large amounts of such dipeptides are highly useful, but tend to have a strong unpleasant flavor.

The total amount of PO and OG in the collagen peptides is preferably 0.05 to 10 wt %, more preferably 0.5 to 5.0 wt %.

The concentrations of the above dipeptides can be measured by a known method, for example, using a device such as LC-MS/MS. A liquid oral composition containing collagen peptides having an average molecular weight of 300 to 2000 and having a total amount of PO and OG in the above range tends to have particularly a strong medium-like odor and a strong bitterness. The present invention can reduce the medium-like odor and bitterness attributable to such collagen peptides in a liquid oral composition containing such collagen peptides.

In the liquid oral composition of the present invention, preferably, the amount of the collagen peptides is 600 to 20000 mg/100 mL. When the amount of the collagen peptides is in the above range, the above-described effect of the present invention can be more sufficiently exerted. When the amount of the collagen peptides in the liquid oral composition is less than 600 mg/100 mL, the medium-like odor and bitterness attributable to the collagen peptides may be mild, and the effect of the present invention to reduce the medium-like odor and bitterness may be less significant.

The amount of the collagen peptides in the liquid oral composition is more preferably 1000 mg/100 mL or more, still more preferably 2000 mg/100 mL or more, while it is more preferably 10000 mg/100 mL or less, still more preferably 7500 mg/100 mL or less. In one embodiment, the amount of the collagen peptides in the liquid oral composition is more preferably 1000 to 10000 mg/100 mL, still more preferably 2000 to 7500 mg/100 mL. The amount means the total amount when several types of collagen peptides are used.

### <Welan Gum>

The welan gum used in the present invention contains polysaccharides obtained from a culture medium of bacteria of the genus Sphingomonas sp. as the main component. Commercially available welan gum can be used. Example of the commercially available welan gum include VISTOP® W available from San-Ei Gen F.F.I., Inc.

In the present invention, adding welan gum to the liquid oral composition containing collagen peptides can reduce the unpleasant odor, particularly medium-like odor, and bitterness attributable to the collagen peptides in the liquid oral composition. Thus, the present invention can provide a liquid oral composition containing collagen peptides in which the collagen peptides are low molecular weight collagen peptides having high absorbability in the body, and the medium-like odor and bitterness are reduced for easy intake.

Preferably, the amount of the welan gum in the liquid oral composition is 50 to 400 mg/100 mL. When the amount of the welan gum is in the above range, the medium-like odor and bitterness attributable to the collagen peptides can be reduced. The amount of the welan gum in the above range is also preferred because when the liquid oral composition is provided as a beverage (beverage composition), the beverage has a drinkable viscosity. The amount of the welan gum in the liquid oral composition is more preferably 80 mg/100 mL or more, still more preferably 100 mg/100 mL or more, for further reducing the medium-like odor and bitterness attributable to the collagen peptides. The amount of the welan gum in the liquid oral composition is more preferably 375 mg/100 mL or less, still more preferably 300 mg/100 mL or less, yet still more preferably 250 mg/100 mL or less, particularly preferably 200 mg/100 mL or less, for making the viscosity of the liquid oral composition more suitable for drinking. In one embodiment, in order to reduce the medium-like odor and bitterness attributable to the collagen peptides and to achieve a viscosity suitable for drinking, the amount of the welan gum in the liquid oral composition is preferably 80 to 375 mg/100 mL, more preferably 80 to 300 mg/100 mL, still more preferably 100 to 250 mg/100 mL, particularly preferably 100 to 200 mg/100 mL.

In order to reduce the unpleasant odor, particularly medium-like odor and bitterness attributable to the collagen peptides, the weight ratio of the collagen peptides to the welan gum (collagen peptides/welan gum) is preferably 1 or more, more preferably 3 or more, still more preferably 10 or more, yet still more preferably 20 or more, particularly preferably 25 or more, while it is preferably 70 or less, more preferably 60 or less, still more preferably 55 or less. In view of the above, in one embodiment, the weight ratio of the collagen peptides to the welan gum (collagen peptides/welan gum) is preferably 1 to 70, more preferably 3 to 70, still more preferably 10 to 60, yet still more preferably 20 to 55, particularly preferably 25 to 55.

### <Other Components and the Like>

The liquid oral composition of the present invention may contain one or more components other than those described above as long as the effect of the present invention is not impaired.

Preferably, the liquid oral composition of the present invention contains a sweetener. Adding a sweetener can add adequate sweetness, providing a better flavor to the liquid oral composition. Any sweetener may be used. Examples include sugars, sugar alcohols, and high-intensity sweeteners. These can be used alone or in combination of two or more thereof.

Examples of the sugars include monosaccharides, disaccharides, trisaccharides, and higher polysaccharides (including oligosaccharide). Specific examples include glucose, fluctose, galactose, mannose, sucrose, maltose, lactose, and trehalose. Examples of the sugar alcohols include erythritol, xylitol, sorbitol, mannitol, maltitol, and reduced palatinose. Of these, erythritol is more preferred. The amount (total amount) of sugar and sugar alcohol in the liquid oral composition is preferably 1000 to 15000 mg/100 mL, more preferably 3000 to 10000 mg/100 mL, for adding adequate sweetness and providing a better flavor.

The high-intensity sweeteners mean sweeteners having higher sweetness than sugars. Specific examples include acesulfame potassium (acesulfame K), sucralose, aspartame, stevia, saccharin, sodium saccharin, and neotame. Of these, acesulfame K and sucralose are preferred. The amount of a high-intensity sweetener in the liquid oral composition is preferably 1 to 50 mg/100 mL, more preferably 3 to 30 mg/100 mL, for adding adequate sweetness and providing a better flavor.

In one embodiment, the liquid oral composition of the present invention preferably contains a sugar or a sugar alcohol and a high-intensity sweetener, and more preferably contains acesulfame K, sucralose, and erythritol. Adding these sweeteners enhances the flavor of the liquid oral composition.

In addition to the collagen peptides, the liquid oral composition of the present invention may also contain other biofunctional materials such as a material known to have a skin improving effect. Examples of the material known to have a skin improving effect include proteoglycans, elastin peptides, ceramides, plant extracts, chondroitin sulfates, glucosamines, minerals (e.g., calcium), and vitamins (e.g., L-ascorbic acid (vitamin C)).

In one embodiment, the liquid oral composition preferably contains a proteoglycan and/or elastin peptide. Proteoglycans and elastin peptide each have a distinct odor in some cases. According to the present invention, when the liquid oral composition contains a proteoglycan and/or elastin peptide in addition to the collagen peptides, it is possible to reduce the odor attributable to the proteoglycan and/or elastin peptide in addition to the medium-like odor and bitterness attributable to the collagen peptides.

The proteoglycan is a general term for compounds in which glycosaminoglycans (mucopolysaccharides) such as chondroitin sulfate and dermatan sulfate are covalently bonded to the core protein. Proteoglycans are found in connective tissues in cartilage and skins of animals, and are essential substances to maintain the structure of these tissues.

The type, source, and production method of the proteoglycan used in the present invention are not limited. For example, proteoglycans extracted from cartilage of fish such as shark, salmon, or ray can be used. Of these, proteoglycans extracted from salmon, particularly from nasal cartilage of salmon, is preferred. Such a proteoglycan can be used alone or in combination with proteoglycans from other sources. Commercially available proteoglycans may be used.

The amount of the proteoglycan in the liquid oral composition of the present invention is preferably 1 to 200 mg/100 mL, more preferably 5 to 100 mg/100 mL. The amount means the total amount when several types of proteoglycans are used.

In the present invention, the elastin peptide means water-soluble elastin peptide. Examples of the elastin peptide include those extracted from biological tissues of animals such as bovine, swine, chicken, sheep, and fish, and decomposition products obtained by, for example, hydrolyzing water-soluble or water-insoluble elastin with an enzyme, acid, alkali, or the like. Artificially synthesized elastin peptide may also be used. One type thereof may be used alone or two or more types thereof may be used.

The molecular weight of the elastin peptide used in the present invention is not limited. Elastin peptides of any molecular weight can be used.

Commercially available elastin peptide may be used. Examples include “Bonito Elastin” (Hayashikane Sangyo Co., Ltd.), “BIDAN Elastin FI” (Nippon Suisan Kaisha, Ltd.), “Tuna Elastin HS-1” (Hagoromo Foods Corporation), and “P-Elastin” (NH Foods Ltd.).

In the liquid oral composition of the present invention, the amount of the elastin peptide is preferably 10 to 750 mg/100 mL, more preferably 50 to 300 mg/100 mL. The amount means the total amount when several types of elastin peptide are used.

In addition to those mentioned above, the liquid oral composition of the present invention may also contain additives such as acidulants, antioxidants, stabilizers, preservatives, flavorings, emulsifiers, pigments, seasonings, pH adjusters, and nutritional enhancer as long as the effect of the present invention is not impaired.

The liquid oral composition of the present invention contains an aqueous medium (usually water). Preferably, the liquid oral composition of the present invention is a liquid oral composition (aqueous liquid oral composition) that uses water as a medium.

The term “liquid” in the liquid oral composition of the present invention means the liquid state at room temperature. The liquid composition is preferably a fluid having a viscosity (22° C.) of about 500 mPa·s or less. In the present invention, the viscosity of the liquid oral composition is the viscosity at 22° C., and can be measured using a B-type viscometer by a method described in Examples.

The viscosity of the liquid oral composition of the present invention is preferably 30 mPa·s or more, preferably 30 to 500 mPa·s. The medium-like odor and bitterness attributable to the collagen peptides can be reduced when the liquid oral composition contains welan gum and having a viscosity in the above range. The liquid oral composition having a viscosity in the above range has a drinkable viscosity when provided as a beverage. The viscosity is more preferably 35 mPa·s or more in order to further reduce the medium-like odor and the like attributable to the collagen peptides. In order to achieve a viscosity more suitable for drinking, the viscosity is more preferably 250 mPa·s or less. In order to reduce the medium-like odor and bitterness attributable to the collagen peptides and to achieve a viscosity suitable for drinking, the viscosity of the liquid oral composition is more preferably 30 to 250 mPa·s, still more preferably 35 to 250 mPa·s. In one embodiment, the amount of the welan gum in the liquid oral composition is preferably an amount that achieves a viscosity in the above range.

In order to provide an antiseptic effect, the pH of the liquid oral composition of the present invention is preferably 5.0 or lower, more preferably 3.0 to 4.5, still more preferably 3.3 to 4.0. The pH herein is the pH at 25° C. An acidulant can be used to adjust the pH. The acidulant is preferably an acid or its salt usable for food and beverages. Examples include acids such as citric acid, phosphoric acid, lactic acid, malic acid, tartaric acid, succinic acid, fumaric acid, gluconic acid, phytic acid, and their salts. One acidulant may be used alone or two or more acidulants may be used in combination. The salt is not limited. Examples include a sodium salt, a potassium salt, and a calcium salt. Only acids in the free acid form or only their salts may be used as acidulants, or free acids and salts may be used in combination. In terms of the flavor of the liquid oral composition, the acidulant preferably includes citric acid or its salt, more preferably citric acid or its salt, or citric acid or its salt and phosphoric acid or its salt.

The amount of the acidulant can be set according to, for example, the type of the acidulant. For example, the amount of acid or its salt used as the acidulant in the liquid oral composition is preferably 100 to 3000 mg/100 mL, more preferably 300 to 2000 mg/100 mL, as the total amount of the acid or its salt in terms of the free acid content. The amount means the total amount when several types of acidulants are used. In regard to the expression “the amount in terms of the free acid content” or its similar expression used herein, when an acid is in the free acid form, such an expression means the free acid content, and when the acid is in the salt form, such an expressions means the value obtained by multiplying the number of moles of the salt by the molecular weight of the corresponding free acid.

The method of producing the liquid oral composition of the present invention is not limited. For example, the method preferably includes a mixing step of mixing the components.

In the mixing step, preferably, the components are mixed by adding an aqueous medium. The aqueous medium is usually water. The order of mixing the components is not limited as long as the components are mixed uniformly. In one embodiment, when a volatile component (e.g., flavoring) or an easily degradable component (e.g., vitamin C) is added, preferably, such a component is added last. The method of producing the liquid oral composition may include, for example, a viscosity adjusting step of adjusting the viscosity. The viscosity adjusting step can be performed by adding welan gum to the composition. The viscosity adjusting step may be performed simultaneously with or after the mixing step. When adding a powdered raw material to the composition containing welan gum, preferably, a solution of the powdered raw material is added to the composition, for facilitating the work to uniformly dissolve the raw material in the composition. The method of producing the liquid oral composition may include a step such as a pH adjusting step of adjusting the pH.

The liquid oral composition of the present invention is preferably used as a beverage (beverage composition).

The liquid oral composition of the present invention can be packed in a container. The form of the container is not limited. A sealed container such as a bottle, can, plastic bottle, paper pack, aluminum pouch, or plastic pouch can be used to pack the liquid oral composition to provide a beverage in a container (packaged beverage) or the like.

### <Method of Improving Flavor of Liquid Oral Composition Containing Collagen Peptides>

The present invention also encompasses a method of improving the flavor of a liquid oral composition containing collagen peptides, the method including adding welan gum to a liquid oral composition containing collagen peptides having an average molecular weight of 300 to 2000.

Adding welan gum to the liquid oral composition containing collagen peptides can reduce the unpleasant odor, particularly medium-like odor, and bitterness attributable to the collagen peptides, and can improve the flavor of the composition. In one embodiment of the method of the present invention, improving the flavor may be reducing the medium-like odor and bitterness.

The method and timing of adding welan gum are not limited as long as the liquid oral composition containing collagen peptides ultimately contains welan gum. The collagen peptides, welan gum, their preferred embodiments, amounts, and the like are the same as those in the liquid oral composition described above. The liquid oral composition may contain other components such as the sweeteners described above. A preferred viscosity of the liquid oral composition containing welan gum is also the same as the preferred viscosity of the liquid oral composition described above. In one embodiment, preferably, welan gum is added to allow the liquid oral composition to have a viscosity in the above range.

### EXAMPLES

The following shows examples that specifically describe the present invention, but the present invention is not limited to these examples.

Raw materials used in the examples and comparative examples are as follows.

Collagen peptides: average molecular weight of 500, extracted from fish
Welan gum: VISTOP® W available from San-Ei Gen F.F.I., Inc. Proteoglycans: Proteoglycan F (product name) available from Ichimaru Pharcos Co. Ltd.
Elastin peptide: Bonito Elastin (product name) available from Hayashikane Sangyo Co., Ltd.
Xanthan gum: San Ace E-S(product name) available from San-Ei Gen F.F.I., Inc.

The above raw materials were used unless otherwise stated.

The average molecular weight of the collagen peptides was measured by a relative molecular mass measurement method in Chinese National Standards (GB standards) GB/T 22729-2008: oligopeptides powder of marine fish. Reagents for M, 451 and M, 189 were glycine-glycine-tyrosine-arginine (M, 451) and glycine-glycine-glycine (M, 189), respectively.

The total amount of Pro-Hyp (PO) and Hyp-Gly (OG) in the above collagen peptides was about 1.4 wt %.

### Examples 1 to 4

Liquid oral compositions (solutions) of Examples 1 to 4 were prepared according to the formulation shown in Table 1, in order to evaluate the effect of reducing the unpleasant flavor derived from collagen peptides. Water was used as a medium.

Specifically, collagen peptides and welan gum were added to and dissolved in water to give a total of 1000 mL, and liquid oral compositions of Examples 1 to 4 were thus obtained.

### Comparatives Examples 1 and 2

Liquid oral compositions were produced by the same method as that of Example 1, except that the raw materials were used in the amounts shown in Table 1. A beverage of Comparative Example 2 contained xanthan gum used as a thickener.

The liquid oral compositions obtained in Examples 1 to 4 and Comparative Examples 1 and 2 were subjected to sensory evaluation of the flavor (odor and taste) at room temperature by a method described below. The viscosity of each liquid oral composition was measured by a method described below. Table 1 shows the formulation of each liquid oral composition and evaluation results.

### <Flavor Evaluation>

Five expert panelists sensorily evaluated the flavor of each liquid oral composition (room temperature) in terms of unpleasant flavor (specifically, medium-like odor (peptone-like odor) and bitterness) based on the following criteria. The results were given based on the average score of the evaluation as follows: 4.0 to 3.1: good; 3.0 to 2.1: fair; less than 2.1: poor.

### Criteria for Flavor

4: Hardly any unpleasant flavor
3: Slight unpleasant flavor
2: Unpleasant flavor
1: Very unpleasant flavor

### <Viscosity Measurement>

The viscosity of each sample (liquid oral composition) was measured using a B-type viscometer by a method described below.

Measurement device (B-type viscometer): BII-type viscometer (Toki Sangyo Co., Ltd.), model BMII

### Rotor: No. 2

Sample container: 100-mL sample bottle
Amount of sample: 75 mL

Each sample was maintained at 22° C. The value was read one minute after the start of rotation, and regarded as the viscosity. The rotational speed during the measurement was 60 rpm.

TABLE 1

| | | | | | Comparative | Comparative |
|---|---|---|---|---|---|---|
| | Example 1 | Example 2 | Example 3 | Example 4 | Example 1 | Example 2 |
| (A) Collagen peptides | 50000 | 50000 | 50000 | 50000 | 50000 | 50000 |
| (B) Welan gum | 750 | 1500 | 2000 | 2500 | — | — |
| Xanthan gum | — | — | — | — | — | 1000 |
| | mg/1000 mL | | | | | |
| (A)/(B) | 66.7 | 33.3 | 25 | 20 | — | — |
| Viscosity (mPa · s) | 34.5 | 96.0 | 156.0 | 222.0 | 3.0 | 35.5 |
| Flavor | Fair | Good | Good | Good | Poor | Poor |

The “(A)/(B)” in Table 1 and Tables 2 and 3 (shown later) is the weight ratio of (A) collagen peptides to (B) welan gum (collagen peptides/welan gum).

The amount of each component in Tables 1 to 3 is the amount (mg/1000 mL) in 1000 mL of the liquid oral composition.

The liquid oral composition of Comparative Example 1 had a very strong medium-like odor and a very strong bitterness derived from the collagen peptides. The liquid oral composition of Comparative Example 2 also had a very strong medium-like odor and a very strong bitterness derived from the collagen peptides as in Comparative Example 1. In the liquid oral compositions of the examples, the medium-like odor and bitterness derived from the collagen peptides were reduced (suppressed). In the liquid oral compositions of Example 2 to 4, the medium-like odor and bitterness derived from the collagen peptides were particularly effectively reduced. The liquid oral compositions of Examples 1 to 4 each also had an easily drinkable viscosity.

### Examples 5 to 8

Liquid oral compositions of Examples 5 to 8 were prepared according to the formulation shown in Table 2 by the same method as that of Example 1. The flavor of each obtained liquid oral composition was evaluated at room temperature by the same method as that of Example 1. The viscosity of each liquid oral composition was measured by the above method. Table 2 shows the evaluation results.

Also in the liquid oral compositions of Examples 5 to 8, adding welan gum reduced the medium-like odor and bitterness derived from the collagen peptides and reduced such an unpleasant flavor.

TABLE 2

| | Example 5 | Example 6 | Example 7 | Example 8 |
|---|---|---|---|---|
| (A) Collagen peptides | 2000 | 6000 | 20000 | 75000 |
| (B) Welan gum | 1500 | 1500 | 1500 | 1500 |
| | mg/1000 mL | | | |
| (A)/(B) | 1.3 | 4.0 | 13.3 | 50.0 |
| Viscosity (mPa · s) | 93.5 | 94.5 | 98.0 | 119.0 |
| Flavor | Good | Good | Good | Fair |

### Examples 9 and 10

Liquid oral compositions of Examples 9 and 10 were prepared according to the formulation shown in Table 3 by the same method as that of Example 1. The flavor of each obtained liquid oral composition was evaluated at room temperature by the same method as that of Example 1. The viscosity of each liquid oral composition was measured by the above method. Table 3 shows the evaluation results.

Also in Examples 9 and 10, adding welan gum reduced the medium-like odor and bitterness derived from the collagen peptides. The proteoglycan and elastin peptide had a fishy flavor. In the liquid oral compositions of Examples 9 and 10 to which welan gum was added, the fishy flavor derived from the proteoglycan and elastin peptide was also reduced.

TABLE 3

| | Example 9 | Example 10 |
|---|---|---|
| (A) Collagen peptides | 50000 | 50000 |
| (B) Welan gum | 1500 | 1500 |
| Proteoglycan | 500 | — |
| Elastin peptide | — | 1500 |

mg/1000 mL

| | | |
|---|---|---|
| (A)/(B) | 33.3 | 33.3 |
| Viscosity (mPa · s) | 101.5 | 101.0 |
| Flavor | Good | Good |

### Example 11

Collagen peptides having an average molecular weight of 931 were used. A liquid oral composition of Example 11 was prepared according to the formulation shown in Table 4 by the same method as that of Example 1. The amount of each component in Table 4 is the amount (mg/1000 mL) in 1000 mL of the beverage. The “(A)/(B)” in Table 4 is the weight ratio of (A) collagen peptides to (B) welan gum (collagen peptides/welan gum). The flavor of the obtained liquid oral composition was evaluated at room temperature by the same method as that of Example 1. The viscosity of the liquid oral composition was evaluated by the above method. Table 4 shows the evaluation results.

### Comparative Examples 3 and 4

The collagen peptides having an average molecular weight of 931 used in Example 11 were used. Liquid oral compositions were produced by the same method as that of Example 11, except that the raw materials were used in the amounts shown in Table 4, and was evaluated in the same manner. Table 4 shows the evaluation results.

TABLE 4

| | | Comparative | Comparative |
|---|---|---|---|
| | Example 11 | Example 3 | Example 4 |
| (A) Collagen peptides | 50000 | 50000 | 50000 |
| (B) Welan gum | 1500 | — | — |
| Xanthan gum | — | — | 1875 |
| | mg/1000 mL | | |
| (A)/(B) | 33.3 | — | — |
| Viscosity (mPa · s) | 85.0 | 5.0 | 94.0 |
| Flavor | Good | Poor | Poor |

The liquid oral compositions of Comparative Examples 3 and 4 each had a very strong medium-like odor and a very strong bitterness derived from the collagen peptides. Adding welan gum reduced the medium-like odor and bitterness derived from collagen peptides.

### INDUSTRIAL APPLICABILITY

The present invention is useful in the food and beverage field and the like.

1. A liquid oral composition, comprising:
a collagen peptide having an average molecular weight of 300 to 2000; and
welan gum.

2. The liquid oral composition according to claim 1,
wherein the amount of the collagen peptide is 600 to 20000 mg/100 mL.

3. The liquid oral composition according to claim 1,
wherein the weight ratio of the collagen peptide to the welan gum (collagen peptide/welan gum) is 3 to 70.

4. The liquid oral composition according to claim 1,
wherein the amount of the welan gum is 50 to 400 mg/100 mL.

5. The liquid oral composition according to claim 1,
wherein the viscosity is 30 to 500 mPa·s.

6. The liquid oral composition according to claim 1, further comprising a proteoglycan and/or elastin peptide.

7. The liquid oral composition according to claim 1,
which is a beverage.

8. A method of improving the flavor of a liquid oral composition, the method comprising:
adding welan gum to a liquid oral composition containing a collagen peptide having an average molecular weight of 300 to 2000.
